(19) United States
(12) Patent Application Publication
(10) Pub. No.: US 2007/0269018 A1
(43) Pub. Date: Nov. 22, 2007

(54) Systems and methods for generating a diffraction profile

(76) Inventors: Geoffrey Harding, Hamburg (DE); Johannes Delfs, Hamburg (DE); Adrian Paul Harding, Hamburg (DE)

Correspondence Address: PATRICK W. RASCHE (22697);ARMSTRONG TEASDALE LLP, ONE METROPOLITAN SQUARE, SUITE 2600, ST. LOUIS, MO, 63102-2740, US

(21) Appl. No.: 11/416,526

(22) Filed: May 3, 2006

(51) Int. Cl. A61B 6/08

(52) U.S. Cl. 378/205

(57) ABSTRACT

A method for aligning an x-ray tube is described. The method includes attaching a frame of the x-ray tube to a first outer side of a gantry of an imaging system by fitting a fastener to a hole formed within the gantry.

### BACKGROUND OF THE INVENTION

This invention relates generally to systems and methods for generating a diffraction profile and more particularly to systems and methods for aligning an x-ray tube.

A conventional x-ray imaging apparatus includes a detector and an x-ray generator. The x-ray generator emits a beam. The beam emitted from the x-ray generator passes through a luggage within an image pickup area and is incident upon the detector. A plurality of detection signals are generated by the detector upon receiving the beam and sent to a data storage apparatus from the detector, stored in the data storage apparatus, and processed by a data processing apparatus. Data obtained by processing the signals is reproduced as an image on a display.

When the x-ray generator is replaced by a new x-ray device due to a failure of the x-ray generator, the x-ray device is aligned with the detector by an alignment procedure. If the x-ray generator is not aligned with respect to the detector, a diffraction peak in a diffraction profile becomes broad, thus making a recognition of an explosive material within the luggage difficult. If the alignment procedure is used, an application of the alignment procedure consumes a long amount of time, is complicated, and is therefore, undesirable.

### BRIEF DESCRIPTION OF THE INVENTION

In one aspect, a method for aligning an x-ray tube is described. The method includes attaching a frame of the x-ray tube to a first outer side of a gantry of an imaging system by fitting a fastener to a hole formed within the gantry.

In another aspect, a system is described. The system includes an x-ray tube including a frame attached to a first outer side of a gantry of an imaging system by fitting a fastener to a hole formed within the gantry.

In yet another aspect, a system for generating a diffraction profile of a substance is described. The system includes a gantry and an x-ray tube including a frame attached to an outer side of the gantry by fitting a fastener to a hole formed within the gantry. The x-ray tube is configured to generate an x-ray beam. The system further includes a detector configured to detect the x-ray beam.

### BRIEF DESCRIPTION OF THE DRAWINGS

FIG. 1 is a block diagram of a system for generating a diffraction profile.

FIG. 2 is a block diagram of an embodiment of the system of FIG. 1.

FIG. 3 a block diagram of another embodiment of a system for generating a diffraction profile.

FIG. 4 is a view of an embodiment of an x-ray tube connected to a gantry of the system of FIG. 3.

FIG. 5 is a perspective view of an embodiment of the x-ray tube.

FIG. 6 is a view of an embodiment of a frame of the x-ray tube.

### DETAILED DESCRIPTION OF THE INVENTION

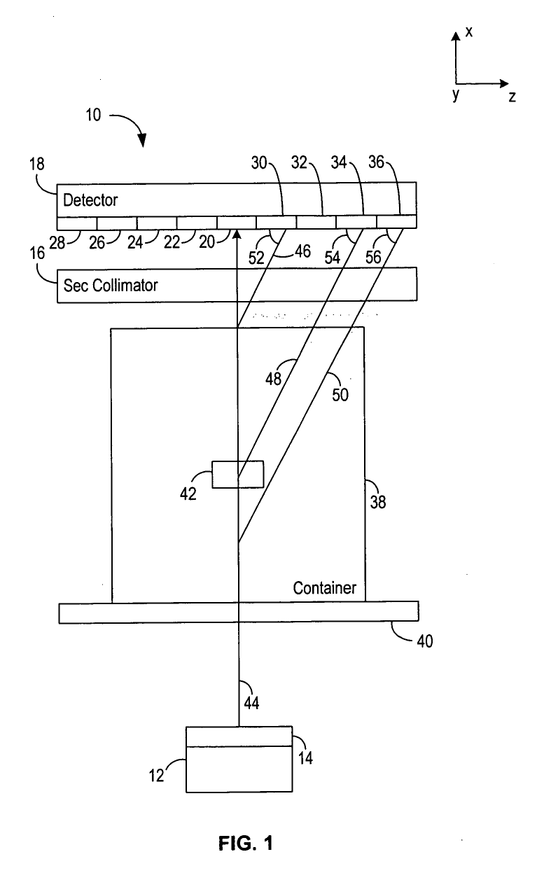

FIG. 1 is a block diagram of a system 10 for generating a diffraction profile of a substance. System 10 includes an x-ray source 12 that includes a primary collimator 14. System 10 further includes a secondary collimator (Sec collimator) 16, and a detector 18. Detector 18 includes a central detector element 20 or a central detector cell for detecting primary radiation. Detector 18 also includes a plurality of detector cells or detector elements 22, 24, 26, 28, 30, 32, 34, and 36 for detecting coherent scatter. Detector 18 includes any number, such as, ranging from and including 256 to 1024, of detector elements. A container 38 is placed on a support 40 between x-ray source 12 and detector 18. Examples of container 38 include a bag, a box, and an air cargo container. Examples of x-ray source 12 include a polychromatic x-ray tube. Container 38 includes a substance 42. Examples of substance 42 include an organic explosive, an amorphous substance having a crystallinity of less than twenty five percent, a quasi-amorphous substance having a crystallinity at least equal to twenty-five percent and less than fifty percent, a partially crystalline substance having a crystallinity at least equal to fifty percent and less than one-hundred percent, and a crystalline substance having a crystallinity of one-hundred percent. Examples of the amorphous, quasi-amorphous, and partially crystalline substances include a gel explosive, a slurry explosive, an explosive including ammonium nitrate, and a special nuclear material. Examples of the special nuclear material include plutonium and uranium. Examples of support 40 include a table and a conveyor belt. An example of detector 18 includes a segmented detector fabricated from Germanium.

X-ray source 12 emits x-rays. Using primary collimator 14, a primary beam 44, such as a pencil beam, is formed from the x-rays generated. Primary beam 44 passes through container 38 arranged on support 40 to generate scattered radiation, such as a plurality of scattered rays 46, 48, and 50. Above support 40, there is arranged detector 18, which measures an intensity of primary beam 44 and photon energy of the scattered radiation. Detector 18 measures the x-rays in an energy-sensitive manner by outputting a plurality of electrical output signals linearly dependent on a plurality of energies of x-ray quanta detected from within primary beam 44 and the scattered radiation.

Detector elements 20, 22, 24, 26, 28, 30, 32, 34, and 36 are geometrically arranged so that a scatter or incident angle of the scatter radiation detected by each detector element 20, 22, 24, 26, 28, 30, 32, 34, and 36 is constant. For example, a scatter angle 52 at which scattered ray 46 is incident on detector element 30 is equal to a scatter angle 54 at which scattered ray 48 is incident on detector element 34 and scatter angle 54 is equal to a scatter angle 56 at which scattered ray 50 is incident on detector element 36. As another example, scattered ray 46 is parallel to scattered rays 48 and 50. Central detector element 20 measures an energy or alternatively an intensity of primary beam 44 after primary beam 44 passes through container 38. Detector elements 22, 24, 26, 28, 30, 32, 34, and 36 separately detect the scattered radiation received from container 38.

Secondary collimator 16 is located between support 40 and detector 18. Secondary collimator 16 includes a number of collimator elements, such as sheets, slits, or laminations, to ensure that the scatter radiation arriving at detector 18 have constant scatter angles and that a position of detector 18 permits a depth in container 38 at which the scatter radiation originated to be determined. The number of collimator elements provided is equal to or alternatively greater than a number of detector elements 20, 22, 24, 26, 28, 30, 32, 34, and 36 and the collimator elements are arranged such that the scattered radiation between neighboring collimator elements each time is incident on one of the detector elements 22, 24, 26, 28, 30, 32, 34, and 36. The collimator elements are made of a radiation-absorbing material, such as, a copper alloy or a silver alloy. In one embodiment employing a fan-beam geometry, a plurality of origination points, within container 38, of the scatter radiation are detected by the detector elements 22, 24, 26, and 28, aligned in a first direction and detector elements 30, 32, 34, and 36 aligned in a second direction that is opposite to and parallel to the first direction. Detector 18 detects the scattered radiation to generate a plurality of electrical output signals. In an alternative embodiment, system 10 does not include primary and secondary collimators 14 and 16.

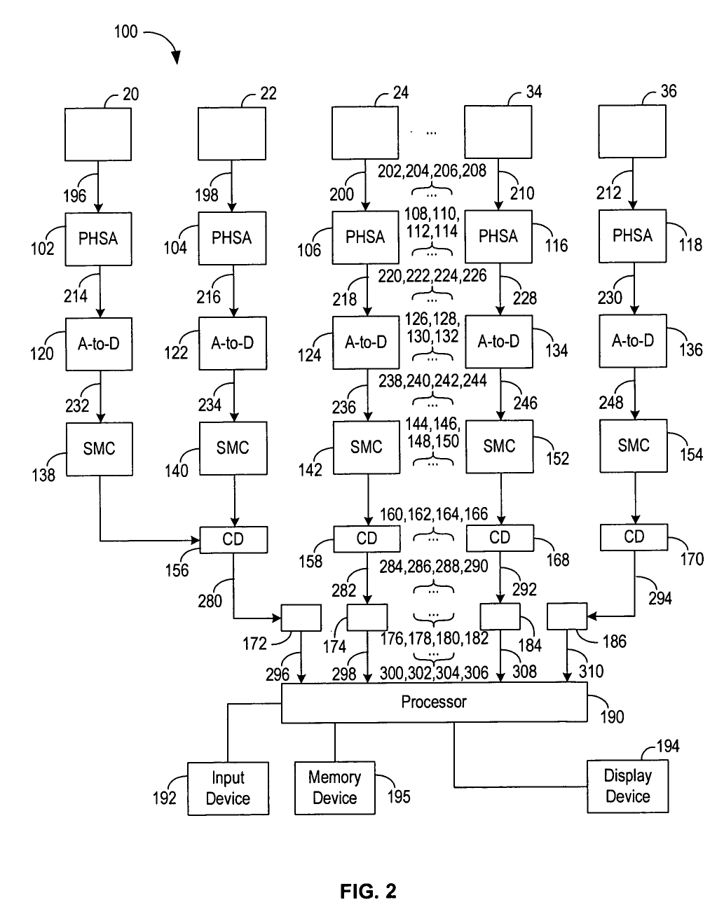

FIG. 2 is a block diagram of an embodiment of a system 100 for generating a diffraction profile of a substance. System 100 includes central detector element 20, detector elements 22, 24, 26, 28, 30, 32, 34, and 36, a plurality of pulse-height shaper amplifiers (PHSA) 102, 104, 106, 108, 110, 112, 114, 116, and 118, a plurality of analog-to-digital (A-to-D) converters 120, 122, 124, 126, 128, 130, 132, 134, and 136, a plurality of spectrum memory circuits (SMCs) 138, 140, 142, 144, 146, 148, 150, 152, and 154 allowing pulse height spectra to be acquired, a plurality of correction devices (CDs) 156, 158, 160, 162, 164, 166, 168, and 170, a plurality of memory devices 172, 174, 176, 178, 180, 182, 184, and 186, a processor 190, an input device 192, a display device 194, and a memory device 195. As used herein, the term processor is not limited to just those integrated circuits referred to in the art as a processor, but broadly refers to a computer, a microcontroller, a microcomputer, a programmable logic controller, an application specific integrated circuit, and any other programmable circuit. The computer includes a device, such as, a floppy disk drive or CD-ROM drive, for reading data including the methods for determining generating a diffraction profile of a substance from a computer-readable medium, such as a floppy disk, a compact disc-read only memory (CD-ROM), a magneto-optical disk (MOD), or a digital versatile disc (DVD). In another embodiment, processor 190 executes instructions stored in firmware. Examples of display device 194 include a liquid crystal display (LCD) and a cathode ray tube (CRT). Examples of input device 192 include a mouse and a keyboard. Examples of each of memory devices 172, 174, 176, 178, 180, 182, 184, 186, and 195 include a random access memory (RAM) and a read-only memory (ROM). An example of each of correction devices 156, 158, 160, 162, 164, 166, 168, and 170 include a divider circuit. Each of spectrum memory circuits 138, 140, 142, 144, 146, 148, 150, 152, and 154 include an adder and a memory device, such as a RAM or a ROM.

Central detector element 20 is coupled to pulse-height shaper amplifier 102, and detector elements 22, 24, 26, 28, 30, 32, 34, and 36 are coupled to pulse-height shaper amplifiers 104, 106, 108, 110, 112, 114, 116, and 118, respectively. Central detector element 20 generates an electrical output signal 196 by detecting primary beam 44 and detector elements 22, 24, 26, 28, 30, 32, 34, and 36 generate a plurality of electrical output signals 198, 200, 202, 204, 206, 208, 210, and 212 by detecting the scattered radiation. For example, detector element 22 generates electrical output signal 198 for each scattered x-ray photon incident on detector element 22. Each pulse-height shaper amplifier amplifies an electrical output signal received from a detector element. For example, pulse-height shaper amplifier 102 amplifies electrical output signal-196 and pulse-height shaper amplifier 104 amplifies electrical output signal 198. Pulse-height shaper amplifiers 102, 104, 106, 108, 110, 112, 114, 116, and 118 have a gain factor determined by processor 190.

An amplitude of an electrical output signal from a detector element is proportional to an energy of an x-ray quantum that is detected by the detector element to generate the electrical output signal. For example, an amplitude of electrical output signal 196 is proportional to an energy of an x-ray quantum in primary beam 44 detected by detector element 20. On the other hand, an amplitude of electrical output signal 198 is proportional to an energy of an x-ray quantum within the scattered radiation that is detected by detector element 22.

A pulse-height shaper amplifier generates an amplified output signal by amplifying an electrical output signal generated from a detector element. For example, pulse-height shaper amplifier 102 generates an amplified output signal 214 by amplifying electrical output signal 196 and pulse-height shaper amplifier 104 generates an amplified output signal 216 by amplifying electrical output signal 198. Similarly, a plurality of amplified output signals 218, 220, 222, 224, 226, 228, and 230 are generated. An analog-to-digital converter converts an amplified output signal from an analog form to a digital form to generate a digital output signal. For example, analog-to-digital converter 120 converts amplified output signal 214 from an analog form to a digital format to generate a digital output signal 232. Similarly, a plurality of digital output signals 234, 236, 238, 240, 242, 244, 246, and 248 are generated by analog-to-digital converters 122, 124, 126, 128, 130, 132, 134, and 136, respectively. A digital value of a digital output signal generated by an analog-to-digital converter represents an amplitude of energy or alternatively an amplitude of intensity of a pulse of an amplified output signal. Each pulse is generated by an x-ray quantum, such as an x-ray photon. For example, a digital value of digital output signal 234 output by analog-to-digital converter 122 is a value of an amplitude of a pulse of amplified output signal 216.

An adder of a spectrum memory circuit adds a number of pulses in a digital output signal. For example, when analog-to-digital converter 122 converts a pulse of amplified output signal 216 into digital output signal 234 to determine an amplitude of the pulse of amplified output signal 216, an adder within spectrum memory circuit 140 increments, by one, a value within a memory device of spectrum memory circuit 140. Accordingly, at an end of an x-ray examination of substance 42, a memory device within a spectrum memory circuit stores a number of x-ray quanta detected by a detector element. For example, a memory device within spectrum memory circuit 142 stores a number of x-ray photons detected by detector element 24 and each of the x-ray photons has an amplitude of energy or alternatively an amplitude of intensity that is determined by analog-to-digital converter 124.

A correction device receives a number of x-ray quanta that have a range of energies and are stored within a memory device of one of spectrum memory circuits 140, 142, 144, 146, 148, 150, 152, and 154, and divides the number by a number of x-ray quanta having the range of energies received from a memory device of spectrum memory circuit 138. For example, correction device 156 receives a number of x-ray photons having a range of energies from a memory device of spectrum memory circuit 140, and divides the number by a number of x-ray photons having the range received from a memory device of spectrum memory circuit 138. Each correction device outputs a correction output signal that represents a range of energies within x-ray quanta received by a detector element. For example, correction device 156 outputs a correction output signal 280 representing an energy spectrum or alternatively an intensity spectrum within x-ray quanta detected by detector element 22. As another example, correction device 158 outputs correction output signal 282 representing an energy spectrum within x-ray quanta detector element 24. Similarly, a plurality of correction output signals 284, 286, 288, 290, 292, and 294 are generated by correction devices 160, 162, 164, 166, 168, and 170, respectively.

Processor 190 receives correction output signals 280, 282, 284, 286, 288, 290, 292, and 294 to generate a momentum transfer x, measured in inverse nanometers (nm⁻¹), from an energy spectrum r(E) of energy E of x-ray quanta within the scattered radiation detected by detector 18. Processor 190 generates the momentum transfer x by applying
x=(E/hc)sin(θ/2) (1)

where c is a speed of light, h is Planck's constant, θ represents constant scatter angles of x-ray quanta of the scattered radiation detected by the detector 18. Processor 190 relates the energy E to the momentum transfer x by equation (1). Mechanical dimensions of the secondary collimator 16 define the scatter angle θ. The secondary collimator 16 restricts the scatter radiation that does not have the angle θ. Processor 190 receives the scatter angle θ from a user via input device 192. Processor 190 generates a diffraction profile of substance 42 by calculating a number of x-ray photons that are detected by detector 18 and by plotting the number versus the momentum transfer x.

It is noted that a number of pulse-height shaper amplifiers 102, 104, 106, 108, 110, 112, 114, 116, and 118 changes with a number of detector elements 20, 22, 24, 26, 28, 30, 32, 34, and 36. For example, five pulse-height shaper amplifiers are used for amplifying signals received from five detector elements. As another example, four pulse-height shaper amplifiers are used for amplifying signals received from four detector elements. Similarly, a number of analog-to-digital converters 120, 122, 124, 126, 128, 130, 132, 134, and 136 changes with a number of detector elements 20, 22, 24, 26, 28, 30, 32, 34, and 36 and a number of spectrum memory circuits 138, 140, 142, 144, 146, 148, 150, 152, and 154 changes with the number of detector elements 20, 22, 24, 26, 28, 30, 32, 34, and 36.

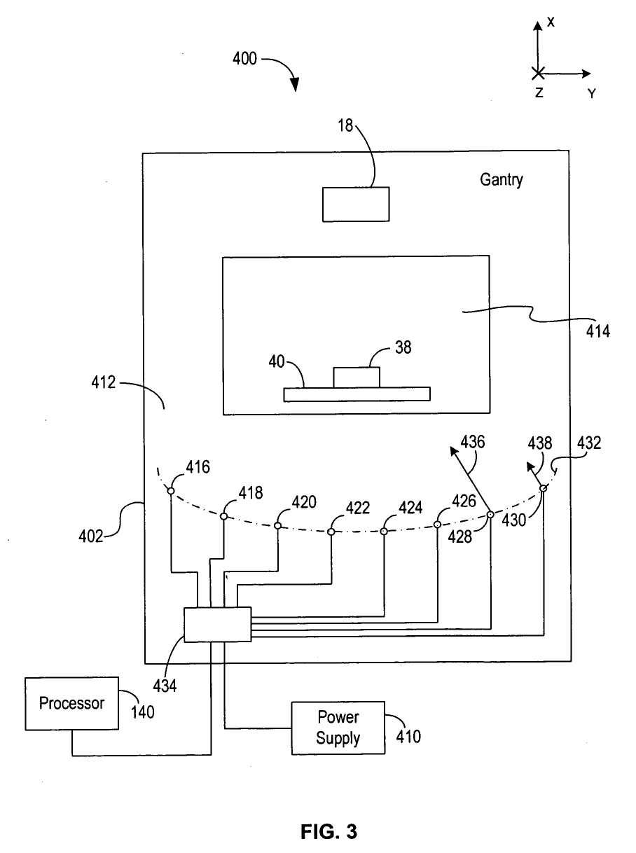

FIG. 3 is a block diagram of an embodiment of a system 400 for generating a diffraction profile. System 400 includes a gantry 402, a power supply 410, and processor 190. Gantry 402 has a side 412. Side 412 is an outer side of gantry 402. An example of gantry 402 includes a gantry having a height, in an x-direction parallel to an x-axis, ranging from and including 2.25 meters to 2.75 meters, a width, in a y-direction parallel to a y-axis, ranging from and including 1.5 meters to 2 meters, and a depth, in a z-direction parallel to a z-axis, ranging from and including 50 centimeters (cm) to 1.5 meters. Gantry 402 has an opening 414 that extends through gantry 402 in the z-direction. An example of opening 414 includes an opening having a height, in the x-direction, ranging from and including 0.75 meters to 1.25 meters and a width, in the y-direction, ranging from and including 1.25 meters to 1.75 meters, and a depth, in the z-direction, that is the same as the depth of gantry 402.

Gantry 402 includes detector 18, a plurality of x-ray tubes 416, 418, 420, 422, 424, 426, 428, and 430, which are an example of x-ray source 12. As an example, a diameter of each of x-ray tubes 416, 418, 420, 422, 424, 426, 428, and 430 ranges from and including 40 millimeters (mm) to 80 mm, and a distance between centers of any two adjacent ones of x-ray tubes 416, 418, 420, 422, 424, 426, 428, and 430 ranges from and including 60 mm to 120 mm. X-ray tubes 416, 418, 420, 422, 424, 426, 428, and 430 are arranged parallel to an arc 432. An example of arc 432 includes an arc having a radius ranging from and including 2 meters to 2.25 meters and an arc length ranging from and including 1.4 meters to 1.8 meters. A center of detector 18 is located at a center of a circle having arc 432.

Gantry 402 further includes an x-ray generation control unit 434 that includes a pulse generator (not shown) that is coupled to processor 190 and that receives power from power supply 410. Power supply 410 is coupled to x-ray tubes 416, 418, 420, 422, 424, 426, 428, and 430 to supply power to x-ray tubes 416, 418, 420, 422, 424, 426, 428, and 430.

Processor 190 issues a command, such as a first on command, a second on command, a first off command, and a second off command. Upon receiving the first on command from processor 190, the pulse generator generates a pulse and transmits the pulse to x-ray tube 428. Upon receiving a pulse from the pulse generator, x-ray tube 428 generates an x-ray beam 436, such as primary beam 44, under a potential applied by power supply 410. Similarly, upon receiving the first off command signal from processor 190, the pulse generator stops transmitting a pulse to x-ray tube 428 and x-ray tube 428 stops generating x-ray beam 436. Furthermore, upon receiving the second on command signal from processor 190, the pulse generator generates and transmits a pulse to any one of the remaining x-ray tubes 416, 418, 420, 422, 424, 426, and 430 and any one of the remaining x-ray tubes 416, 418, 420, 422, 424, 426, and 430 generates an x-ray beam. For example, upon receiving the second on command signal from processor 190, the pulse generator generates and transmits a pulse to x-ray tube 430 and x-ray tube 430 generates an x-ray beam 438. Upon receiving the second off command signal from processor 190, the pulse generator stops transmitting a pulse to any one of the remaining x-ray tubes 416, 418, 420, 422, 424, 426, and 430 and the one of the remaining x-ray tubes 416, 418, 420, 422, 424, 426, and 430 stops generating an x-ray beam. It is noted that in an alternative embodiment, system 400 includes a higher number, such as 10 or 20, or alternatively a lower number, such as 4 or 6, of x-ray tubes than that shown in FIG. 3.

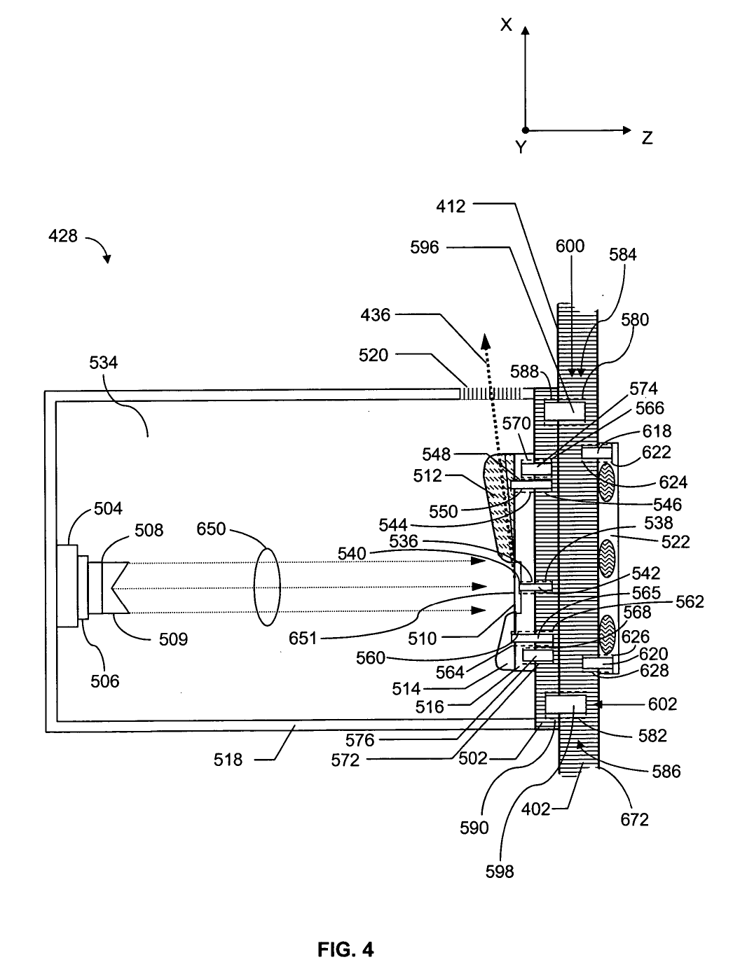

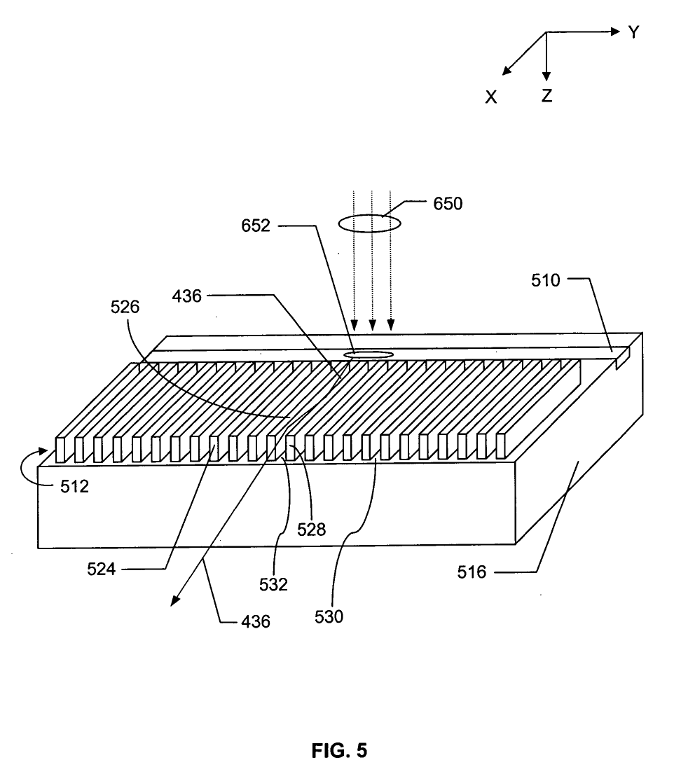

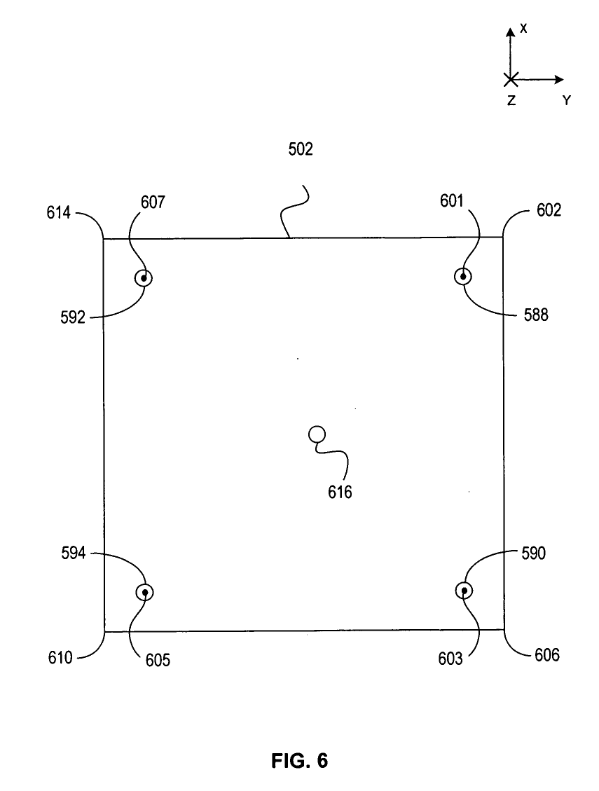

FIG. 4 is a view of an embodiment of x-ray tube 428 connected to gantry 402, FIG. 5 is a perspective view of an embodiment of x-ray tube 428, and FIG. 6 is a view of an embodiment of a frame 502 of x-ray tube 428. Remaining x-ray tubes 416, 418, 420, 422, 424, 426, and 430 are similar in construction to x-ray tube 428. X-ray tube 428 includes an insulating layer 504, a cathode 506, a grid electrode 508, an electron optical element 509, an anode 510, a collimator 512, a shaping shim 514, a heat spreader 516, frame 502, a tube housing 518, and an x-ray window 520. Examples of electron optical element 509 include a plate, a diaphragm, and a shaped conductor. Electron optical element 509 may be fabricated from copper or molybdenum. Collimator 512 forms a portion of primary collimator 14. X-ray tube 428 is coupled to gantry 402 that is coupled to a cooling flange 522. Insulating layer 504 can be a ceramic insulating layer and is attached, such as bolted, to tube housing 518. Cathode 506 is mounted on insulating layer 504 and is fabricated from a thermionic element, such as tungsten. Anode 510 is embedded within heat spreader 516, is positioned with respect to frame 502, and may be fabricated from a tungsten-rhenium alloy. An example of anode 510 includes a strip of length ranging from and including 50 mm to 80 mm in the y-direction, a height ranging from and including 9 mm to 11 mm in the x-direction, and a thickness ranging from and including 450 micrometers (μm) to 1000 μm in the z-direction. Shaping shim 514 is fabricated from a material having a high melting point, such as a melting point from and including 2000 degrees Fahrenheit (° F.) to 4000° F. Examples of the material used to fabricate shaping shim 514 include tungsten, tantalum, and molybdenum.

Collimator 512 includes a plurality of collimator walls, including collimator walls 524, 526, and 528, which are fabricated from tantalum or alternatively tungsten. As an example, each of collimator walls of collimator 512 have a width ranging from and including 0.75 mm to 1.25 mm in the y-direction, a length ranging from and including 90 mm to 110 mm in the x-direction, and a depth ranging from and including 250 μm to 1000 μm in the z-direction. A plurality of channels, such as channel 530 and channel 532 formed between collimator walls 526 and 528, formed between collimator walls of collimator 512 converge at the center of detector 18. An example of heat spreader 516 includes a heat spreader fabricated from a metal, such as copper, aluminum, or silver. Frame 502 may be fabricated from a stable metal, such as steel or molybdenum. X-ray window 520 is fabricated from beryllium or aluminum. An example of tube housing 518 includes a housing fabricated from steel.

A vacuum chamber 534 is located within tube housing 518. A vacuum is created within vacuum chamber 534 by a vacuum pump (not shown) via an exhaust port (not shown). Vacuum chamber 534 includes insulating layer 504, grid electrode 508, cathode 506, electron optical element 509, collimator 512, shaping shim 514, anode 510, and heat spreader 516. Tube housing 518, x-ray window 520, and frame 502 enclose vacuum chamber 534. A seal is formed between a plurality of sides of x-ray window 520 and a plurality of sides of tube housing 518 that are adjacent to the sides of x-ray window 520 to secure an air-tightness of x-ray tube 428 and maintain a vacuum within vacuum chamber 534. Cooling flange 522 is located on a side 672 of gantry 402 that is in a direction opposite to the z-direction of side 412 of gantry 402 that is coupled to frame 502. Side 672 is an outer side of gantry 402.

A user, such as a person, connects anode 510 to heat spreader 516 and to frame 502 by drilling a hole 536 extending into and not through heat spreader 516, drilling a hole 538 extending into and not through frame 502, and drilling a hole 540 extending into and not through anode 510, and fitting a fastener 542 into holes 536, 538, and 540. The user uses a drilling machine to drill a hole. The user aligns holes 536, 538, and 540 with each other in the z-direction. A laser pointer is used to align a plurality of holes in the z-direction. Examples of a fastener includes a lug and a pin, such as a dowel pin. Alternatively, the user fits anode 510 with heat spreader 516 and frame 502 by using any number, such as 2 or 3, holes formed within each of anode 510, heat spreader 516, and frame 502, and fitting the number of fasteners within the holes.

The user attaches collimator wall 528 to heat spreader 516 and frame 502 by drilling a hole 544 extending through heat spreader 516, drilling a hole 546 extending into and not through frame 502, and drilling a hole 548 extending into and not through collimator wall 528, and fitting a fastener 550 into holes 546, 548, and 550. The user aligns holes 546, 548, and 550 with each other in the z-direction. Similarly, the user fits each of the remaining collimator walls of collimator 512 with heat spreader 516 and frame 502. For example, the user attaches collimator wall 530 to heat spreader 516 and frame 502 by drilling a hole extending through heat spreader 516, drilling a hole extending into and not through frame 502, and drilling a hole extending into and not through collimator wall 530, and fitting a fastener into the holes. In an alternative embodiment, the user attaches any of collimator walls of collimator 512 to heat spreader 516 and frame 502 by a number, such as 2 or 4, of holes formed within each of the collimator walls, heat spreader 516, and frame 502, and by fitting the number of fasteners into the holes.

The user couples shaping shim 514 to heat spreader 516 and frame 502 by drilling a hole 560 extending through heat spreader 516, a hole 562 extending into and not through frame 502, and a hole 564 extending into and not through shaping shim 514, and fitting a fastener 565 into holes 560, 562, and 564. The user aligns holes 560, 562, and 564 with each other in the z-direction. Alternatively, the user attaches shaping shim 514 to heat spreader 516 and to frame 502 via a number, such as 2 or 4, of holes, formed within each of shaping shim 514, heat spreader 516, and frame 502, and by fitting the number of fasteners into the holes.

The user fits heat spreader 516 within frame 502 by drilling a plurality of holes 566 and 568 extending into and not through frame 502, drilling a plurality of holes 570 and 572 extending into and not through heat spreader 516, fitting a faster 574 into holes 566 and 570, and fitting a fastener 576 into holes 568 and 572. The user aligns holes 566 and 570 with each other in the z-direction and aligns holes 568 and 572 with each other in the z-direction. Alternatively, the user attaches heat spreader 516 to frame 502 via a higher number, such as 4 or 5, of holes formed within each of heat spreader 516 and frame 502, and the higher number of fasteners than that shown in FIG. 4. In another alternative embodiment, the user attaches heat spreader 516 to frame 502 via a lower number, such as 1, of holes formed within each of heat spreader 516 and frame 502, and the lower number of fasteners than that shown in FIG. 4.

The user uses an oven to heat x-ray tube 428 to remove gaseous impurities within vacuum chamber 428. The user attaches x-ray tube 428 to gantry 402 by attaching frame 502 to gantry 402. The user attaches frame 502 to side 412 of gantry 402 by drilling a plurality of holes 580, 582, 584 (not visible), and 586 (not visible) extending into and not through gantry 402, drilling a plurality of holes 588, 590, 592, and 594 extending into and not through frame 502, and fitting a plurality of fasteners 596, 598, 600 (not visible), and 602 (not visible) within holes 580, 582, 584, 586, 588, 590, 592, and 594. For example, the user fits fastener 596 within holes 580 and 588, fits fastener 598 within holes 582 and 590, fits fastener 600 within holes 584 and 592, and fits fastener 602 within holes 586 and 594. As another example, the user drills hole 588 having a center 601 at a distance, such as ranging from and including 9 mm to 11 mm, from an edge 602 of frame 502, drills hole 590 having a center 603 at the distance from an edge 606 of frame 502, drills hole 594 having a center 605 at the distance from an edge 610 of frame 502, and drills hole 592 having a center 607 at the distance from an edge 614 of frame 502. Alternatively, as an example, when a frame that is fitted by the user with gantry 402 and anode 510, is circular in shape, such as having a diameter ranging from and including 50 mm to 70 mm, the user drills each of holes 588, 590, 592, and 594 at the distance from a circumference of the frame. The user drills hole 588 to be diagonally opposite to hole 594 and drills hole 590 to be diagonally opposite to hole 592. Alternatively, the user drills hole 588 to not be diagonally opposite to hole 594 and drills hole 590 to be diagonally opposite to hole 592. In another alternative embodiment, the user drills hole 590 to not be diagonally opposite to hole 592 and drills hole 588 to be diagonally opposite to hole 594. In yet another alternative embodiment, in addition to holes 588, 590, 592, and 594, the user drills a hole 616 to extend through a center of frame 502 into gantry 402, and fits a bolt into hole 616 to attach frame 502 to gantry 402. In still another alternative embodiment, the user fits frame 502 with gantry 402 via a higher number, such as 6 or 7, number of holes formed within each of gantry 402 and frame 502.

The user couples cooling flange 522 to gantry 402 via a plurality of fasteners 618 and 620, and a plurality of holes 622, 624, 626, and 628. The user attaches cooling flange 522 to side 672 of gantry 402 by drilling holes 624 and 628 extending into and not through gantry 402 and drilling holes 622 and 626 extending into and not through cooling flange 522, fitting fastener 618 into holes 622 and 624, and fitting fastener 620 into holes 626 and 628.

The user attaches x-ray tube 428 to gantry 402 by inserting fastener 596 into hole 588, fastener 598 into hole 590, fastener 600 into hole 592, and fastener 602 into hole 594, aligning x-ray tube 428 with gantry 402 to align fastener 596 with hole 580 of gantry 402, to align fastener 598 with hole 582 of gantry 402, to align fastener 600 with hole 584 of gantry 402, and to align fastener 602 with hole 586 of gantry 402. The user attaches x-ray tube 428 to gantry 402 by aligning x-ray tube 428 with gantry 402 and pressing x-ray tube 428 against gantry 402 to insert fastener 596 into hole 580, insert fastener 598 into hole 582, insert fastener 600 into hole 584, and insert fastener 602 into hole 586. X-ray tube 428 is aligned with detector 18 and/or secondary collimator 16 by attaching x-ray tube 428 to gantry 402. A standard deviation of an alignment of x-ray tube 428 with detector 18 is at most 10 μm. Moreover, x-ray tube 428 can be operated to transmit x-ray beam 436 immediately, such as from 1 minute to 30 minutes, after aligning x-ray tube 428 with detector 18 and/or secondary collimator 16. The user aligns x-ray tube 428 with detector 18 and/or secondary collimator 16 by attaching x-ray tube 428 to holes 580, 582, 584, and 586 of gantry 402. Similarly, the user aligns x-ray tubes 416, 418, 420, 422, 424, 426, and 430 with respect to detector 18 and/or secondary collimator 16.

In the event of a failure x-ray tube 428, the user decouples x-ray tube 428 from gantry 402 by pulling x-ray tube 428 in a direction opposite to the z-direction. Fastener 596 does not extend into hole 580, fastener 598 does not extend into hole 582, fastener 600 does not extend into hole 584, and fastener 602 does not extend into hole 586 upon pulling x-ray tube 428 in a direction opposite to the z-direction. X-ray tube 428 is not aligned with detector 18 and secondary collimator 16 when the user decouples x-ray tube 428 from gantry 402. The user attaches a new x-ray tube to gantry 402 in place of x-ray tube 428 in a manner similar to that of attaching x-ray tube 428 to gantry 402. It is noted that a fasteners are fabricated from a metal, such as steel. It is also noted that a diameter of a fastener ranges from and including 9 mm to 11 mm.

Power supply 410 biases cathode 506 by supplying a negative voltage, such as ranging from and including −150 kilovolts (kV) to −250 kV. Moreover, power supply 410 biases anode 510 by grounding anode 510. Additionally, power supply 410 biases grid electrode 508 by supplying a negative voltage, such as ranging from and including −160 kV to −260 kV, to grid electrode 508. For example, if cathode 506 is biased at −150 kV, grid electrode 508 is biased at −160 kV and if cathode 506 is biased at −250 kV, grid electrode 508 is biased at −260 kV. Upon receiving the first on command from processor 190, the pulse generator generates a pulse from power received from power supply 410 and the pulse cancels the negative voltage applied to grid electrode 508 to generate either a zero or a positive potential at grid electrode 508. Upon canceling the negative voltage applied to grid electrode 508, a plurality of electrons within an electron beam 650 travel and accelerate from cathode 506 via grid electrode 508 and electron optical element 509 to anode 510. Electron optical element 509 shapes an electric field around cathode 506 to focus electron beam 650 at anode 510.

Shaping shim 514 shapes an electric field generated from electron beam 650 so that a plurality of electric field lines of the electric field are parallel to a surface 651 of anode 510 facing cathode 506. Anode 510 is heated upon receiving the electrons within electron beam 650. The electrons within electron beam 650 that are incident on anode 510 at a focal spot 652 loose energy by contacting anode 510 to generate the x-rays. The x-rays pass through a plurality of collimator walls, such as collimator walls 526 and 528, of collimator 512. Collimator walls, such as collimator walls 526 and 528, collimate the x-rays to generate x-ray beam 436. Collimator 512 collimates the x-rays to generate x-ray beam 436 having a standard deviation of less than one degree in an xy plane formed by the x and y axes, and less than 0.1 degrees in an xz plane formed by the x and z axes. An x-ray beam is generated by each of x-ray tubes 416, 418, 420, 422, 424, 426, and 430 in a similar manner in which x-ray beam 436 is generated by x-ray tube 428. For example, upon receiving a command signal from processor 190, the second pulse generator generates a pulse from power received from power supply 410 and the pulse cancels a negative voltage applied to a grid electrode within x-ray tube 430 to generate an electron beam. In the example, the electron beam contacts an anode of x-ray tube 430 to generate the x-rays and the x-rays are collimated by a collimator of the x-ray tube 430 to generate x-ray beam 438.

X-ray beam 436 passes through x-ray window 520 of x-ray tube 428 and is incident on container 38. The heat generated within anode 510 when anode 510 comes in contact with electron beam 650 is dissipated by heat spreader 516. Moreover, a cooling fluid, such as a cooling oil or water, is supplied to cooling flange 522 from a supply source (not shown) to cool anode 510 via gantry 402, frame 502, and heat spreader 516. It is noted that in an alternative embodiment, the actions performed by the user can be performed by a fabrication system, such as a robotic system.

Technical effects of the herein described systems and methods for generating a diffraction profile include providing holes within gantry 402 and x-ray tube 428 to allow an alignment of x-ray tube 428 with respect to detector 18 and/or secondary collimator 16. Positions of holes within gantry 402 and x-ray tube 428 may be generated by application of a ray trace simulation program by processor 190. The simulation program generates the positions and orientations of holes to maximize an intensity of a diffraction profile of substance 42, to maximize a spatial resolution of container 38, and to maximize a resolution in the momentum transfer x. X-ray tube 428 is aligned with respect to secondary collimator 16 placed within gantry 402 and/or with respect to detector 18 placed within gantry 402 by fitting x-ray tube 428 to holes of gantry 402. Other technical effects include generating a higher x-ray emission output than that generated by a conventional x-ray tube. The higher x-ray emission output is generated by locating collimator 512 inside vacuum chamber 534 of x-ray tube 428.

While the invention has been described in terms of various specific embodiments, those skilled in the art will recognize that the invention can be practiced with modification within the spirit and scope of the claims.

What is claimed is:

1. A method for aligning an x-ray tube, said method comprising attaching a frame of the x-ray tube to a first outer side of a gantry of an imaging system by fitting a fastener to a hole formed within the gantry.

2. A method in accordance with claim 1 further comprising fitting the fastener within a hole formed within the frame of the x-ray tube.

3. A method in accordance with claim 1 further comprising decoupling the x-ray tube from the gantry by removing the fastener from the hole of the gantry.

4. A method in accordance with claim 1 further comprising decoupling the x-ray tube from the gantry by removing the fastener from the hole of the gantry, wherein said removing the fastener from the hole is performed by pulling the x-ray source in a direction away from the gantry.

5. A method in accordance with claim 1 further comprising attaching a cooling flange on a second outer side of the gantry, wherein the first outer side is located in a direction opposite to a direction of location of the second outer side.

6. A method in accordance with claim 1 further comprising locating a collimator inside a vacuum chamber of the x-ray tube by attaching the collimator to the frame.

7. A method in accordance with claim 1 wherein said attaching the frame of the x-ray tube comprises coupling the frame with the gantry by fitting a pin within the hole of the gantry and a hole of the frame.

8. A method in accordance with claim 1 further comprising attaching a heat spreader of the x-ray tube to the frame of the x-ray tube by fitting a pin within a hole of the frame and within a hole of the heat spreader.

9. A method in accordance with claim 1 further comprising attaching an anode of the x-ray tube to the frame of the x-ray tube by fitting a pin within a hole of a heat spreader, within a hole of the frame of the x-ray tube, and within a hole of the anode.

10. A method in accordance with claim 1 further comprising attaching a collimator wall of the x-ray tube with the frame of the x-ray tube by fitting a pin through a hole of a heat spreader, within a hole of the frame, and within a hole of the collimator wall.

11. A method in accordance with claim 1 further comprising attaching a shaping shim of the x-ray tube with the frame of the x-ray tube by fitting a pin through a hole of a heat spreader, within a hole of the frame, and within a hole of the shaping shim.

12. A system comprising an x-ray tube including a frame attached to a first outer side of a gantry of an imaging system by fitting a fastener to a hole formed within the gantry.

13. A system in accordance with claim 12 wherein the fastener is fitted within a hole formed within the frame of said x-ray tube.

14. A system in accordance with claim 12 wherein said x-ray tube is decoupled from the gantry by removing the fastener from the hole of the gantry.

15. A system in accordance with claim 12 wherein said x-ray tube is decoupled from the gantry by removing the fastener from the hole of the gantry, and wherein the fastener is removed from the hole by pulling tube x-ray tube in a direction away from the gantry.

16. A system in accordance with claim 12 further comprising a cooling flange attached to a second outer side of the gantry, wherein the first outer side is located in a direction opposite to a direction of location of the second outer side.

17. A system for generating a diffraction profile of a substance, said system comprising:
a gantry;
an x-ray tube including a frame attached to an outer side of said gantry by fitting a fastener to a hole formed within said gantry, wherein said x-ray tube configured to generate an x-ray beam; and
a detector configured to detect the x-ray beam.

18. A system in accordance with claim 17 wherein the fastener is fitted within a hole formed within the frame of said x-ray tube.

19. A system in accordance with claim 17 wherein said x-ray tube is decoupled from said gantry by removing the fastener from the hole of said gantry.

20. A system in accordance with claim 17 configured to generate the diffraction profile.
